(19) United States
(12) Patent Application Publication
(10) Pub. No.: US 2017/0346323 A1
(43) Pub. Date: Nov. 30, 2017

(54) WIRELESS DIGITAL DETECTOR WITH MOTION CHARGING

(71) Applicant: Carestream Health, Inc., Rochester, NY (US)

(72) Inventor: Scott T. MacLaughlin, Pittsford, NY (US)

(21) Appl. No.: 15/163,734

(22) Filed: May 25, 2016

(51) Int. Cl. H02J 7/00; A61B 6/00; H02J 7/34

(52) U.S. Cl. CPC H02J 7/0052; H02J 7/0044; A61B 6/4233; A61B 6/4405; H02J 7/345

(57) ABSTRACT

A portable digital radiography detector has a two dimensional array of photosensors with control electronics electrically connected to the photosensors. A power source is electrically connected to the control electronics. A kinetic charging system is electrically connected to the power source to recharge the power source. There is a substantially rigid housing enclosing the photosensors, the control electronics, and the kinetic charging system.

### FIELD OF THE INVENTION

The invention relates generally to the field of medical imaging and more particularly relates to apparatus and methods for forming an x-ray detector having the capability to convert kinetic energy from motion to electrical energy.

### BACKGROUND

Digital radiography (DR) imaging converts incident x-ray radiation energy to pixellated digital image content using a scintillator material that converts the x-ray energy to light for detection by an array of photodetectors. The portable DR detector has a housing that supports and protects the scintillator material and its accompanying photodetector array and also contains various other types of circuitry for providing power, control, and data communication for the detector. Requirements for packaging of the sensing and support components within the detector housing are demanding. Conventional housing arrangements are characterized by high component count, complex cable routing, and the need for on-board battery power to allow wireless operation. Even with high-capacity portable battery power, the detector is constrained to relatively short operating times and the need to recharge or replace the rechargeable power source. To prevent loss of time during an examination, the operator may be required to bring extra recharged batteries, which may need to be installed, adding to the overhead and complexity of detector use and operation.

Existing methods for providing power to a portable wireless DR detector are workable, but have shortcomings that can limit the effectiveness of these devices. One approach uses a removable battery or other power source. For this type of configuration, the operator typically carries one or more spare batteries to allow on-site replacement. Battery replacement, however, takes time and carrying spare batteries complicates the workflow for operation of the portable radiography system, with added weight and parts that must accompany the technician moving from one site to the next. This can have negative impact on workflow and efficiency. In some cases, replacement can necessitate an initialization cycle or rebooting of the DR detector, delaying progress from one task to the next. There can be other negative aspects of on-site battery replacement, including parts tracking, cost, reliability, operator training, the requirement for external recharging systems, and ongoing maintenance and management.

Another conventional method for providing portable DR detector power eliminates some of the inherent problems with replaceable battery use, but introduces other difficulties. This alternative method provides the DR detector with an integrated power source, such as a battery or storage capacitor that is built into the detector and is not removable by the operator. With an integrated power source, however, the detector must be periodically recharged, effectively removing the detector from usability until recharging is complete.

The disruption and delay that can result from recharging or reloading power sources for a portable DR detector can compromise the ability of practitioners to provide portable imaging services promptly and effectively in hospital and general clinical environments. There would be advantages to solutions that help to extend useful life of the battery in order to increase efficiency and throughput and decrease charging requirements.

### SUMMARY

An aspect of this application is to advance the art of medical digital radiography and to address, in whole or in part, at least the foregoing and other deficiencies of the related art. It is another aspect of this application to provide in whole or in part, at least the advantages described herein. For example, certain exemplary embodiments of the application address the need to extend the useful operating time of on-board battery sources by converting kinetic energy from motion of the detector in transport and handling to electrical charge.

According to one aspect of the disclosure, there is provided a portable digital x-ray detector comprising:
a two dimensional array of photosensors;
control electronics electrically connected to the photosensors;
a power source electrically connected to the control electronics;
a kinetic charging system electrically connected to the power source to recharge the power source; and
a substantially rigid housing enclosing the photosensors, the control electronics, and the kinetic charging system.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is an exploded, perspective view showing components of a DR detector, as packaged within a housing.

FIG. 2 is an exploded, perspective view showing components of a DR detector according to an alternate packaging embodiment.

FIG. 3 is a schematic diagram that shows a DR detector having a motion-charging device in an electrical circuit.

FIG. 4 is an exploded, perspective view showing components of a DR detector having a plurality of motion-charging devices.

FIG. 5 shows shaking or agitating the DR detector to impart kinetic energy for recharging.

FIG. 6 is a schematic diagram that shows a DR detector having a motion-charging device with a super-capacitor in an electrical circuit.

FIG. 7 is a perspective view that shows a mobile digital radiography system that applies vibrational movement to a DR detector during transport.

### DESCRIPTION OF EXEMPLARY EMBODIMENTS

The following is a description of exemplary embodiments, reference being made to the drawings in which the same reference numerals identify the same elements of structure in each of the several figures.

Where they are used in the present disclosure, the terms “first”, “second”, and so on, do not necessarily denote any ordinal, sequential, or priority relation, but are simply used to more clearly distinguish one element or set of elements from another, unless specified otherwise.

As used herein, the term “energizable” relates to a device or set of components that perform an indicated function upon receiving electrical power and, optionally, upon receiving an enabling electrical signal.

In the context of the present disclosure, the phrase “in signal communication” indicates that two or more devices and/or components are capable of communicating with each other via electrical or wireless signals that travel over some type of signal path such as a conductor, a radio wave channel, an optical fiber, or a light wave in free space. The signals may include data communication, power, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

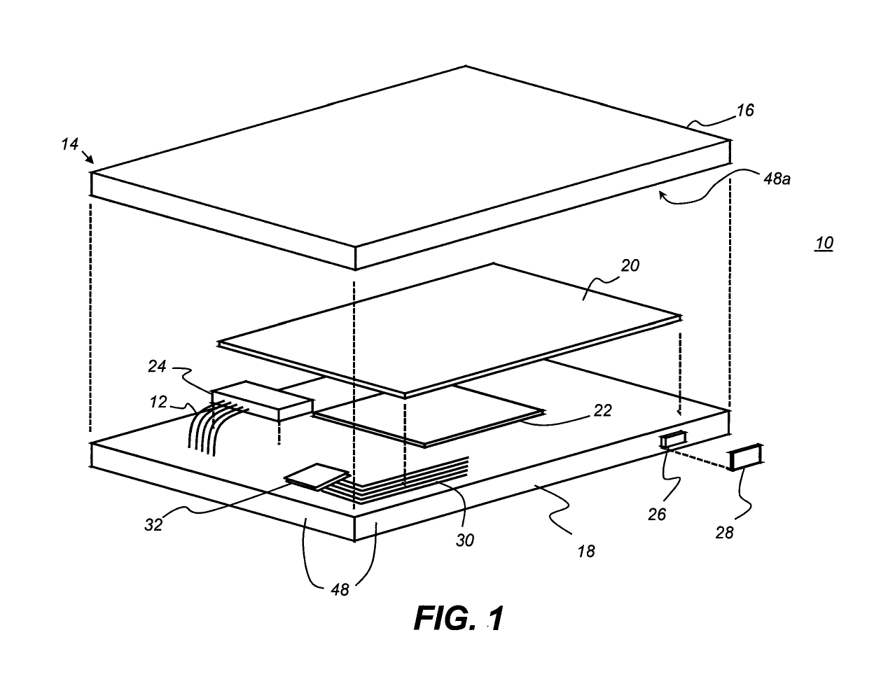

The exploded view of FIG. 1 shows, in simplified form, some of the electrically active internal components of a DR detector 10 that are protected within an enclosure or housing 14 formed using multiple parts, including top and bottom covers 16 and 18. A detector array 20, that may include a scintillator layer, provides a recording medium via a two-dimensional radiation sensitive pixel array for capturing image signals from received radiographic radiation to generate image data. The scintillator generates output light energy when energized by an x-ray exposure. A circuit board 22 provides supporting control electronics components for image data acquisition and wireless transmission to an external host system. A battery 24 provides power, acting as the voltage source for detector 10 operations. A port 26 extending through bottom cover 18 is provided to allow electrical connection for receiving and transmitting data, and/or receiving power such as from a voltage supply. The port may have an optional cover plate or sealing cap 28, which may be a rubber seal or other liquid-proofing material. In addition to the illustrated components, a number of interconnecting cables, supporting fasteners, cushioning materials, connectors, and other elements may be used for packaging and protecting the DR detector circuitry. An optional antenna 30 and transmitter 32 for wireless communication may alternately be provided, with antenna 30 extending within the housing 14. Top and bottom housing covers 16 and 18 may be fastened together along a mating surfaces 48 and 48a. One or more cables 12, such as multi-wire flexible cables, may also be included within housing 14 for interconnection between components.

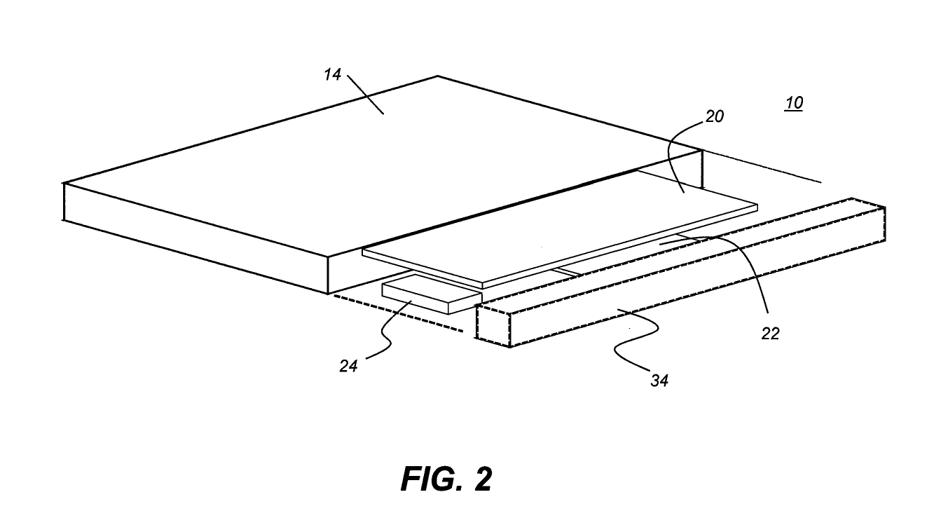

The exploded view of FIG. 2 shows an alternate embodiment of DR detector 10, in which detector array 20, circuit board 22, and battery 24, along with interconnection and other support components, slide into an encased cavity in an enclosure or housing 14 through an open end thereof. A lid 34 may be fastened to housing 14 to provide a protective seal.

The rechargeable battery 24 for the wireless DR detector is typically a Lithium-ion battery (LIB) battery pack, often used for portable electronics devices. Alternately, a storage capacitor can be used for providing portable device power.

Embodiments of the present disclosure address the need for improved operating time by utilizing motion-charging that converts kinetic energy from normal movement of the DR detector 10 in transport and handling. The movement of the detector can come from sources including normal vibration from transport or handling, either when carried by the operator, inserted or removed from a table or wall stand bucky or other device, or when transferred from one site to another in a cart or other transport. Other potential sources of kinetic energy include standard handling, such as when the device is positioned behind or underneath the patient and when the detector is removed and restored to the cart, and purposeful manual or mechanical vibration, shaking, and repetitive back and forth linear or rotational movement.

So-called motion-charging devices that generate electrical charge from kinetic energy are known. In one type of device, inductive energy generated by motion of a magnet within a system of coiled wires is converted into electrical current. Reference is made to US Patent Application Publication No. 2015/0214823 by Shastry et al., which is hereby incorporate by reference herein, and to the publication: “Case Study: AMPY Keeps Mobile Devices Charged - - - and Mobile”, 2015, Proto Labs, Maple Plain Minn.

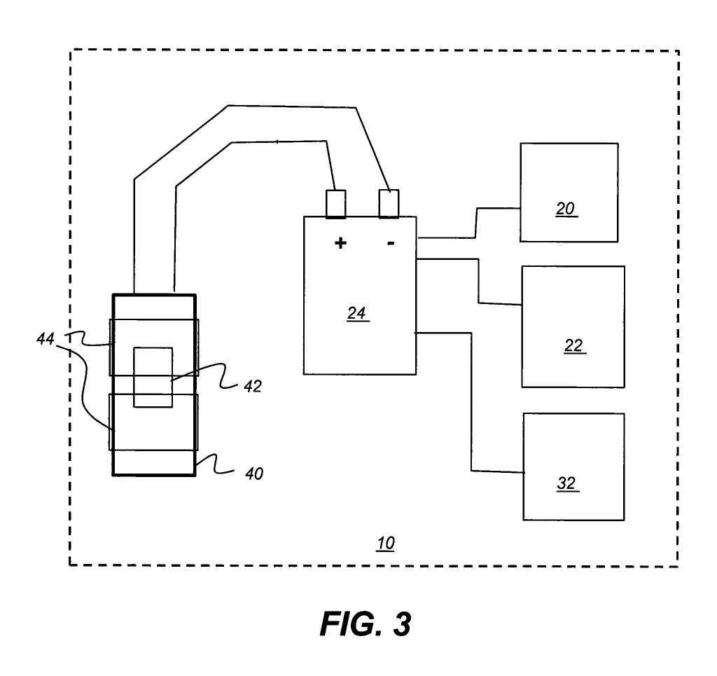

The schematic diagram of FIG. 3 shows DR detector 10 having a motion-charging device 40 installed therein and electrically connected thereto in an electrical circuit. Battery 24 provides power for a number of components, including detector array 20, one or more circuit boards 22, and transmitter 32, for example. Motion-charging device 40 is connected to battery 24 to provide supplementary charging by induction. Within motion-charging device 40, there may be a suspended magnet 42 that interacts with one or more sets of coils 44 for generating electrical energy due to relative motion and/or vibration as between the magnet and coils. One of the magnets 42 or coils 44 may be configured in a fixed position while the other may be movable, or both the magnets 42 and coils 44 may be movable, to thereby covert relative motion into induced electrical current used for charging the battery 24. Additional components can include regulator circuitry and related circuitry for controlling the flow of electric charging current into the battery 24, for example.

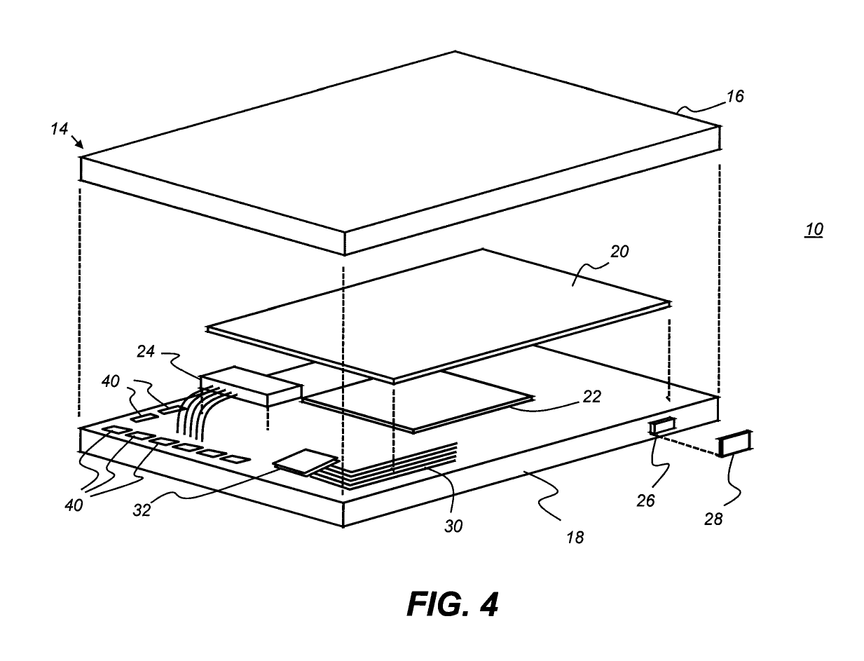

One or more motion-charging devices 40 can be installed within the DR detector housing. The perspective, exploded view of FIG. 4 shows DR detector 10 having a number of motion-charging devices 40, distributed along portions of the detector housing. Assembling the devices 40 along the periphery of the housing can be beneficial and allows these components to use space that is otherwise unused within the housing 14. Motion-charging devices 40 can be arranged to take advantage of kinetic energy in any direction, including kinetic energy from movement in orthogonal directions.

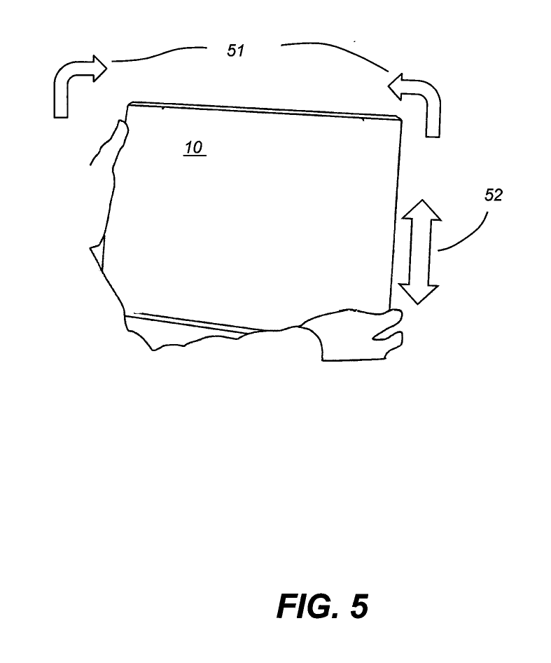

According to an embodiment of the present disclosure, one or more motion-charging devices 40 are MEMS (Micro-ElectroMechanical Systems) devices. It may be convenient for the operator to extend the interval between battery replacement by manually partially rotating in a back-and-forth motion 51 or linearly shaking or agitating the DR detector 52, as shown in FIG. 5, in order to generate sufficient electrical energy for ongoing use. This can be useful, for example, when an operator is not in proximity to a recharging power source, such as when the operator or practitioner is moving about a medical facility on rounds, outside of a hospital setting such as for veterinary x-ray in an outside environment on a farm, or for security applications such as imaging a suspicious package or object on location in the field. Although shaking, agitating, partially rotating or otherwise imparting kinetic energy to the detector would not be the primary method of recharging, it could be used as a back-up method for allowing x-ray acquisition where there is no external recharging power source available, or where time delay is particularly undesirable.

While the lithium-ion battery can provide suitable performance for the portable DR detector, there are some acknowledged shortcomings inherent to use of the Lithium-ion battery as the power source. The detector itself must be designed with an appropriate well or enclosure for seating the battery to allow its removal for recharging. Additional consideration must be provided for optimizing the position of battery contacts, providing suitable battery latches, storing spare batteries, design and maintenance of charging equipment, and overall administration of the battery tracking and recharging process. Li-Ion batteries are relatively expensive to manufacture and can have additional costs due to hazardous material shipping and disposal. Li-ion cells can contain some corrosive materials. Under some conditions, Li-ion batteries can experience thermal runaway, potentially damaging the battery and creating undesirable conditions for nearby equipment.

An embodiment of the present disclosure addresses the need for improved storage performance and significantly reduced recharging time using one or more super-capacitors (SCs) also termed ultra-capacitors. Using nanotechnology materials, the super-capacitor may have charge storage many times the storage capacity of even high-capacitance electrolytic capacitors. These can include capacitors designed using electrochemical principles, such as an electrochemical double-layer, carbon-based capacitors or a pseudo-capacitor formed using nanoparticle structures such as carbon nano-tubes. Commercial supercapacitors for power applications, for example, can exhibit capacitance in excess of 100 Farads. Reference is made to U.S. Patent Application Publication No. 2010/0238607 by Park et al., which is hereby incorporated by reference herein. Reference is also made to an online article entitled “Nanotech Battery Breakthrough Promises 30 Second Smartphone Charging” by Antony Leather, at www.forbes.com, available on Apr. 4, 2014 ( . . . /sites/antonyleather/2014/04/04/new-nanotech-breakthrough-promises-30-second-smartphone-charging/), and to an online article entitled “In D.C. and China, Two Approaches To A Streetcar Unconstrained By Wires” npr cities project, at www.npr.org, available on Oct. 22, 2015 ( . . . /2015/10/22/450583840/in-d-c-and-china-two-approaches-to-a-streetcar-unconstrained-by-wires).

Super-capacitors formed using nanoparticle materials can be charged to full capacity in a very short time, such as within 30 seconds for some types of devices. SC technology has demonstrated a much improved charge/discharge cycle life as compared to a typical Li-Ion battery which can extend the useful life and charge capacity of the detector power source. The size and weight of the nano super capacitor can be similar to a Li-Ion battery. Among benefits of this technology applied to wireless digital detectors are (1) detector cost savings, since the power source can be contained within the detector, there is no need for spare batteries, no need for additional chargers, simpler IPX sealing with no battery well, improved reliability since no battery mating contacts, or battery latch moving parts; (2) manufacturing costs of the proposed power source can be less than traditional Li-Ion batteries, (3) savings in shipping and disposal since the organic compounds used can be non-toxic and environmentally safe, (3) workflow improvements, since recharging can be accomplished in the time it takes to replace a rechargeable battery. There are also materials advantages, as the organic compounds used in the nano-material super capacitor are non-toxic and environmentally safe.

Advantageously, the charge time of a supercapacitor can be less than a minute. The charge characteristic is similar to that used for an electrochemical battery; the charge current is limited largely by the charger's current handling capability. Initial charging can execute quickly, with additional time added for charge completion. Provision must be made to limit the inrush current when charging an empty supercapacitor. The supercapacitor is not subject to overcharge and does not require full-charge detection; the current simply stops flowing when the device is fully charged.

The supercapacitor can be charged and discharged a virtually unlimited number of times. Unlike the electrochemical battery, which has a defined cycle life, the supercapacitor can be repeatedly cycled. The supercapacitor does not tend to age in the same way as the conventional battery. Under normal conditions, a supercapacitor degrades from its original 100 percent capacity to 80 percent in 10 years. Applying higher voltages than specified can tend to shorten SC life span. The SC is forgiving in hot and cold temperatures, an advantage that batteries cannot meet equally well.

The self-discharge of a supercapacitor is substantially higher than that of an electrostatic capacitor and somewhat higher than an electrochemical battery; the organic electrolyte contributes to this. The supercapacitor discharges from 100 to 50 percent in 30 to 40 days. Lead and lithium-based batteries, in comparison, self-discharge about 5 percent per month.

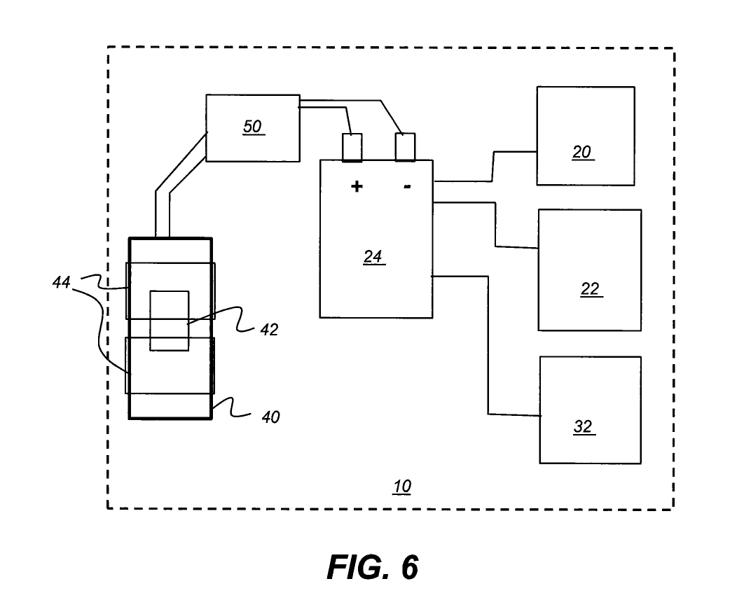

The lithium ion supercapacitor has layers of lithium ions and may use a carbon nanoparticle for its anode. FIG. 6 is a schematic diagram that shows a DR detector having a motion-charging device with a super-capacitor 50 installed therein and connected in an electrical circuit. Here, super-capacitor 50 is electrically connected to an optional battery 24 as part of a hybrid power system, using a motion-charging device 40. Super-capacitor 50 may also be used without a battery connection.

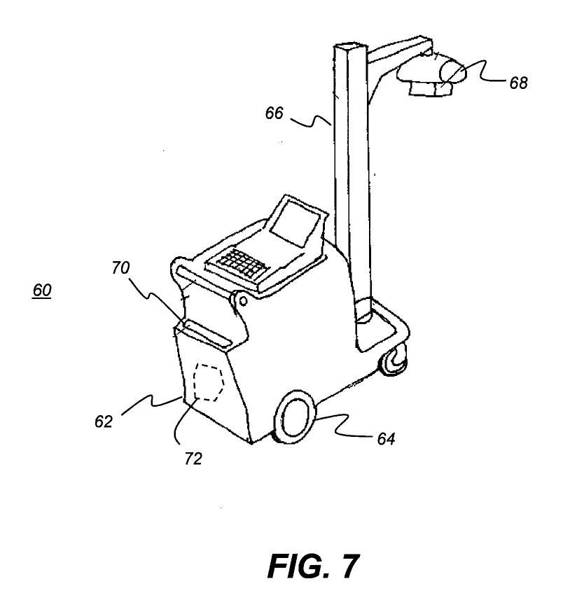

FIG. 7 is a perspective view that shows a mobile digital radiography system 60 according to an embodiment of the present disclosure. Mobile system 60 has a base 62 with wheels 64 for rollably transporting system 60. Base 62 supports a column 66 having an x-ray tube head 68 attached thereto. A slot 70 provides a holding-place for securing the DR detector for the system during storage and transport. An actuator 72, such as an eccentric cam, vibration plate, or other device, provides vibrational energy or other movement to the DR detector during transport, for conversion of kinetic energy to electrical energy. Using a vibration energy source, for example, multiple detectors may be stacked and recharged at the same time. Vibration from transport itself may be sufficient to provide suitable levels of kinetic energy to induce electric current for charging the DR detector as described herein.

The invention has been described in detail, and may have been described with particular reference to a suitable or presently preferred embodiment, but it will be understood that variations and modifications can be effected within the spirit and scope of the invention. In addition, while a feature(s) of the invention can have been disclosed with respect to only one of several implementations/embodiments, such feature can be combined with one or more other features of other implementations/embodiments as can be desired and/or advantageous for any given or identifiable function. The term “at least one of” is used to mean one or more of the listed items can be selected. The term “about” indicates that the value listed can be somewhat altered, as long as the alteration does not result in nonconformance of the process or structure to the illustrated embodiment. Finally, “exemplary” indicates the description is used as an example, rather than implying that it is an ideal. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed:

1. A portable digital radiography detector comprising:
a two dimensional array of photosensors;
control electronics electrically connected to the photosensors;
a power source electrically connected to the control electronics;
a kinetic charging system electrically connected to the power source to recharge the power source; and
a substantially rigid housing enclosing the photosensors, the control electronics, and the kinetic charging system.

2. The detector of claim 1 wherein the kinetic charging system comprises at least one magnet and at least one inductor.

3. The detector of claim 1 wherein the kinetic charging system comprises a plurality of motion-charging devices that provide electrical signals in response to being kinetically energized by forces delivered in orthogonal directions.

4. The detector of claim 1 wherein the power source comprises a super capacitor.

5. The detector of claim 1 wherein the power source comprises a super capacitor and a power storage device electrically connected to the super capacitor, the power storage device comprising layers of lithium ions.

6. A mobile digital radiography system comprising:
a base comprising wheels for rollably transporting the system;
a column attached to the base and having an x-ray tube head attached thereto; and
a slot for securing a digital radiography detector, wherein the detector is configured to capture radiographic images generated by the tube head, and wherein the slot is configured to move back and forth repetitively to deliver kinetic energy to the detector.

7. The system of claim 6 wherein the detector comprises a kinetic energy power source that is configured to recharge in response to the delivered kinetic energy.

8. The system of claim 6 wherein the kinetic energy source is a vibrational energy source.

9. A portable digital radiography detector comprising:
a two dimensional array of photosensors;
control electronics electrically connected to the photosensors;
a power source electrically connected to the control electronics, the power source comprising a capacitor having a capacitance in excess of 100 Farads;
a charging system electrically connected to the power source to recharge the capacitor; and
a substantially rigid housing enclosing the photosensors, the control electronics, and the charging system.

10. The system of claim 9 wherein the power source further comprises a battery.

11. The system of claim 9 wherein the charging system converts kinetic energy to electrical energy.

12. The system of claim 9 wherein the capacitor is formed of layers of lithium ions.

13. A method for charging a portable digital radiography detector, the method comprising shaking or agitating the detector to actuate one or more motion charging devices disposed within a housing of the detector.
